(19) United States
(12) Patent Application Publication
(10) Pub. No.: US 2026/0250263 A1
(43) Pub. Date: Aug. 27, 2026

(54) SOLID STATE FORMS OF ANLOTINIB AND PROCESS FOR PREPARATION THEREOF

(71) Applicant: ASSIA CHEMICAL INDUSTRIES LTD., Tel Aviv (IL)

(72) Inventor: Lorena Kordic, Rab (HR)

(21) Appl. No.: 19/160,133

(22) PCT Filed: Feb. 27, 2024
(86) PCT No.: PCT/IB2024/051869

§ 371 Date: Aug. 27, 2025

Related U.S. Application Data
(60) Provisional application No. 63/448,368, filed on Feb. 27, 2023.

(51) Int. Cl. C07D 401/12; A61K 31/4709

(52) U.S. Cl. CPC C07D 401/12; A61K 31/4709

(57) ABSTRACT

Solid state Form of Anlotinib processes for preparation thereof, and pharmaceutical compositions thereof are described.

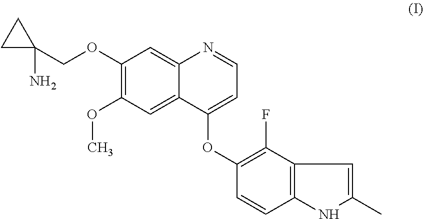 [SMILES: COc1cc2c(Oc3ccc4[nH]c(C)cc4c3F)ccnc2cc1OCC1(N)CC1]

### FIELD OF THE DISCLOSURE

The present disclosure encompasses solid state forms of Anlotinib, in embodiments crystalline polymorphs of Anlotinib or salts thereof, processes for preparation thereof, and pharmaceutical compositions thereof.

### BACKGROUND OF THE DISCLOSURE

Anlotinib, 1-[[4-[(4-fluoro-2-methyl-1H-indol-5-yl)-oxy]-6-methoxyquinolin-7-yl]oxymethyl]cyclopropan-1-amine, has the following chemical structure:

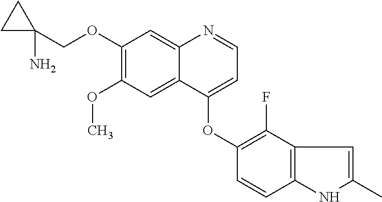 [SMILES: COc1cc2c(Oc3ccc4[nH]c(C)cc4c3F)ccnc2cc1OCC1(N)CC1]

Anlotinib is a tyrosine kinase inhibitor that targets vascular endothelial growth factor receptor (VEGFR), fibroblast growth factor receptor (FGFR), platelet-derived growth factor receptors (PDGFR), and c-kit. Anlotinib may both inhibit angiogenesis and halt tumor cell growth. It is currently developed for the treatment of cancer, e.g., non-small lung cancer (NLSC).

The compound is described in U.S. Pat. No. 8,148,532.

Salts of Anlotinib are described in WO 2016/179123 and WO 2020/001406. Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (13C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Anlotinib or salts thereof.

### SUMMARY OF THE DISCLOSURE

The present disclosure provides crystalline polymorphs of Anlotinib or salts thereof, processes for preparation thereof, and pharmaceutical compositions thereof. These crystalline polymorphs can be used to prepare other solid state forms of Anlotinib, Anlotinib salts and their solid state forms.

The present disclosure also provides uses of the said solid state forms of API in the preparation of other solid state forms of Anlotinib or salts thereof.

The present disclosure provides crystalline polymorphs of Anlotinib or salts thereof for use in medicine, including for the treatment of cancer.

The present disclosure also encompasses the use of crystalline polymorphs of Anlotinib or salts thereof of the present disclosure for the preparation of pharmaceutical compositions and/or formulations.

In another aspect, the present disclosure provides pharmaceutical compositions comprising crystalline polymorphs of Anlotinib or salts thereof according to the present disclosure.

The present disclosure includes processes for preparing the above mentioned pharmaceutical compositions. The processes includes combining any one or a combination of the crystalline polymorphs of Anlotinib or salts thereof with at least one pharmaceutically acceptable excipient.

The crystalline polymorph of Anlotinib or salt thereof as defined herein and the pharmaceutical compositions or formulations of the crystalline polymorph of Anlotinib or salts thereof may be used as medicaments, such as for the treatment of cancer.

The present disclosure also provides methods of treating cancer, by administering a therapeutically effective amount of any one or a combination of the crystalline polymorphs of Anlotinib or salt thereof of the present disclosure, or at least one of the above pharmaceutical compositions, to a subject suffering from cancer, or otherwise in need of the treatment.

The present disclosure also provides uses of crystalline polymorphs of Anlotinib or salts thereof of the present disclosure, or at least one of the above pharmaceutical compositions, for the manufacture of medicaments for treating e.g. cancer.

### BRIEF DESCRIPTION OF THE DRAWINGS

FIG. 1 shows a characteristic X-ray powder diffraction pattern (XRPD) of Anlotinib Form B2;

FIG. 2 shows a characteristic XRPD of Anlotinib Form B4;

FIG. 3 shows a characteristic XRPD of Anlotinib Form B5;

FIG. 4 shows a characteristic XRPD of Anlotinib HCl Form L1;

FIG. 5 shows a characteristic XRPD of Anlotinib HCl Form L2;

FIG. 6 shows a characteristic XRPD of Anlotinib HCl Form L3; and

FIG. 7 shows a characteristic XRPD of Anlotinib HCl Form L3.

### DETAILED DESCRIPTION OF THE DISCLOSURE

The present disclosure encompasses solid state forms of Anlotinib, including crystalline polymorphs of Anlotinib or salts thereof, processes for preparation thereof, and pharmaceutical compositions thereof.

Solid state properties of Anlotinib or Anlotinib salts and crystalline polymorphs thereof can be influenced by controlling the conditions under which Anlotinib and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Anlotinib described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Anlotinib. In some embodiments of the disclosure, the described crystalline polymorph of Anlotinib may contain from about 1% to about 20% (w/w), from about 5% to about 20% (w/w), or from about 5% to about 10% (w/w) of one or more other crystalline polymorph of the same Anlotinib.

A compound may be referred to herein as chemically pure or purified compound or as substantially free of any other compounds. As used herein in this context, the expression “substantially free of any other compounds” will be understood to mean that the pure compound contains about 20% (w/w) or less, about 10% (w/w) or less, about 5% (w/w) or less, about 2% (w/w) or less, about 1% (w/w) or less, or about 0% of any other compound as measured, for example, by HPLC. Thus, pure or purified Anlotinib or Anlotinib salt herein as substantially free of any compounds would be understood to contain greater than about 80% (w/w), greater than about 90% (w/w), greater than about 95% (w/w), greater than about 98% (w/w), greater than about 99% (w/w), or about 100% of the subject Anlotinib. In some embodiments of the disclosure, the described pure or purified Anlotinib may contain from about 1% to about 20% (w/w), from about 5% to about 20% (w/w), or from about 5% to about 10% (w/w) of one or more other compounds.

The solid state form may be referred to herein as “Anlotinib Form B2” or “Crystalline Form B2 of Anlotinib” or “Crystalline Anlotinib Form B2” or “Crystalline polymorph B2 of Anlotinib” or “Crystalline Anlotinib polymorph B2” or “Anlotinib polymorph B2”. For example, crystalline Form B2 of Anlotinib may be interchangeably referred to herein as Anlotinib Form B2 or as Crystalline Anlotinib Form B2 or as Crystalline polymorph B2 of Anlotinib or as Crystalline Anlotinib polymorph B2 or Anlotinib polymorph B2.

Depending on which other crystalline polymorphs a comparison is made, the crystalline polymorphs of Anlotinib or salts thereof of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Anlotinib referred to herein as being characterized by graphical data “as depicted in” or “as substantially depicted in” a Figure will thus be understood to include any crystal forms of Anlotinib characterized with the graphical data having such small variations, as are well known to the skilled person, in comparison with the Figure.

As used herein, and unless stated otherwise, the term “anhydrous” in relation to crystalline forms of Anlotinib, relates to a crystalline form of Anlotinib which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

As used herein, the term “isolated” in reference to crystalline polymorph of Anlotinib of the present disclosure corresponds to a crystalline polymorph of Anlotinib that is physically separated from the reaction mixture in which it is formed.

As used herein, unless stated otherwise, the XRPD measurements are taken using copper Kα radiation wavelength 1.5418 Å. XRPD peaks reported herein are measured using Cuk α radiation, λ=1.5418 Å, typically at a temperature of 25±3° C.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

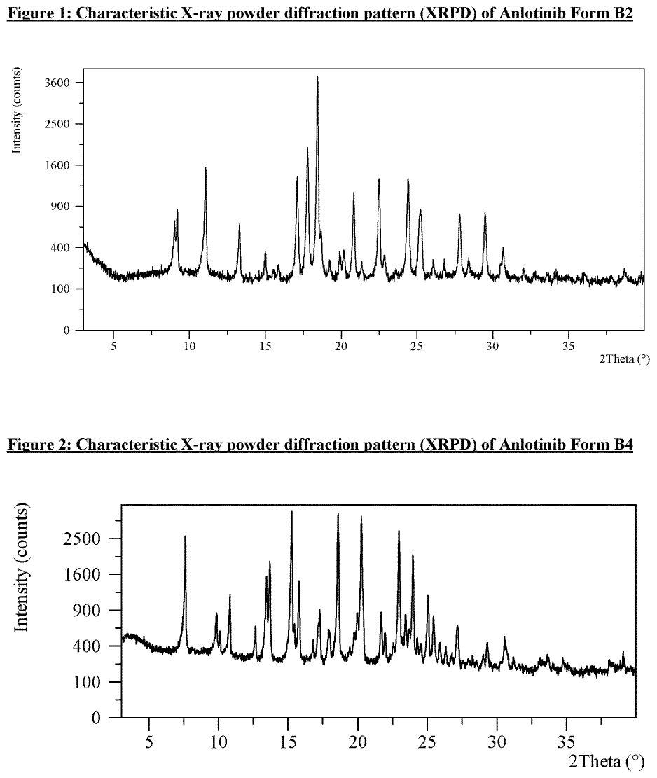

The present disclosure includes a crystalline polymorph of Anlotinib, designated Form B2. The crystalline Form B2 of Anlotinib may be characterized by data selected from one or more of the following: an X-ray powder diffraction pattern substantially as depicted in FIG. 1; an X-ray powder diffraction pattern having peaks at 11.1, 17.1, 17.8, 20.8 and 22.5 degrees 2-theta±0.2 degrees 2-theta; and combinations of these data.

Crystalline Form B2 of Anlotinib may be further characterized by an X-ray powder diffraction pattern having peaks at 11.1, 17.1, 17.8, 20.8 and 22.5 degrees 2-theta±0.2 degrees 2-theta, and also having any one, two, three, four or five additional peaks selected from 9.0, 13.3, 18.5, 24.4 and 29.5 degrees 2-theta±0.2 degrees 2-theta.

In one embodiment of the present disclosure, crystalline Form B2 of Anlotinib is isolated.

Crystalline Form B2 of Anlotinib may be characterized by each of the above characteristics alone/or by all possible combinations, e.g., an XRPD pattern having peaks at 11.1, 17.1, 17.8, 20.8 and 22.5 degrees 2-theta±0.2 degrees 2-theta; an XRPD pattern as depicted in FIG. 1, and combinations thereof.

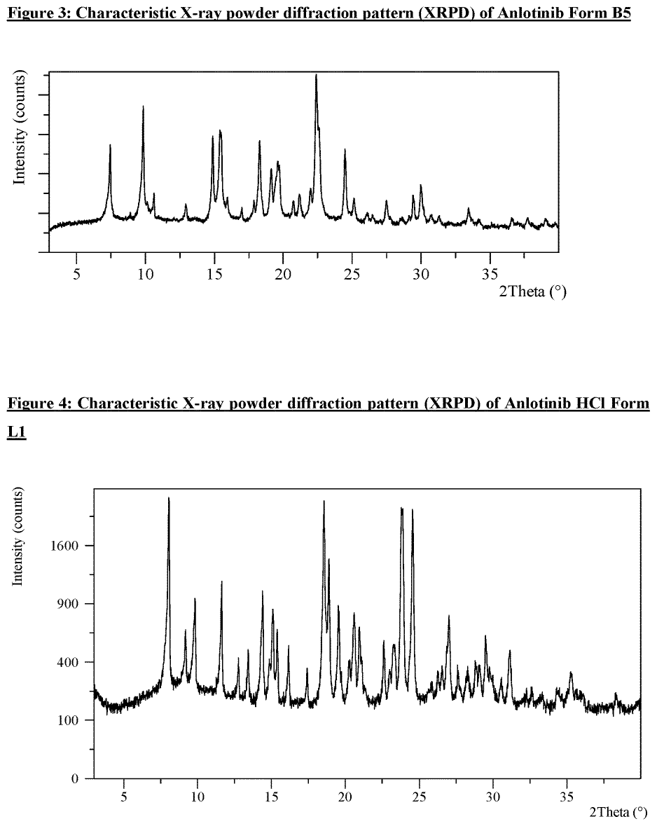

The present disclosure further includes a crystalline polymorph of Anlotinib, designated Form B4. The crystalline Form B4 of Anlotinib may be characterized by data selected from one or more of the following: an X-ray powder diffraction pattern substantially as depicted in FIG. 2; an X-ray powder diffraction pattern having peaks at 7.6, 9.9, 13.7 and 15.3 degrees 2-theta±0.2 degrees 2-theta; and combinations of these data.

Crystalline Form B4 of Anlotinib may be further characterized by an X-ray powder diffraction pattern having peaks at 7.6, 9.9, 13.7 and 15.3 degrees 2-theta±0.2 degrees 2-theta, and also having any one, two, three, four or five additional peaks selected from 13.4, 18.6, 20.3, 23.0 and 24.0 degrees 2-theta±0.2 degrees 2-theta.

In one embodiment of the present disclosure, crystalline Form B4 of Anlotinib is isolated.

Crystalline Form B4 of Anlotinib may be characterized by each of the above characteristics alone/or by all possible combinations, e.g., an XRPD pattern having peaks at 7.6, 9.9, 13.7 and 15.3 degrees 2-theta±0.2 degrees 2-theta; an XRPD pattern as depicted in FIG. 2, and combinations thereof.

The present disclosure further includes a crystalline polymorph of Anlotinib, designated Form B5. The crystalline Form B5 of Anlotinib may be characterized by data selected from one or more of the following: an X-ray powder diffraction pattern substantially as depicted in FIG. 3; an X-ray powder diffraction pattern having peaks at 14.9, 18.3, 19.1, 22.4 and 24.5 degrees 2-theta±0.2 degrees 2-theta; and combinations of these data.

Crystalline Form B5 of Anlotinib may be further characterized by an X-ray powder diffraction pattern having peaks at 14.9, 18.3, 19.1, 22.4 and 24.5 degrees 2-theta±0.2 degrees 2-theta, and also having any one, two, three, four or five additional peaks selected from 7.4, 9.8, 12.9, 27.5 and 30.0 degrees 2-theta±0.2 degrees 2-theta.

In one embodiment of the present disclosure, crystalline Form B5 of Anlotinib is isolated.

Crystalline Form B5 of Anlotinib may be characterized by each of the above characteristics alone/or by all possible combinations, e.g., an XRPD pattern having peaks at 14.9, 18.3, 19.1, 22.4 and 24.5 degrees 2-theta±0.2 degrees 2-theta; an XRPD pattern as depicted in FIG. 3, and combinations thereof.

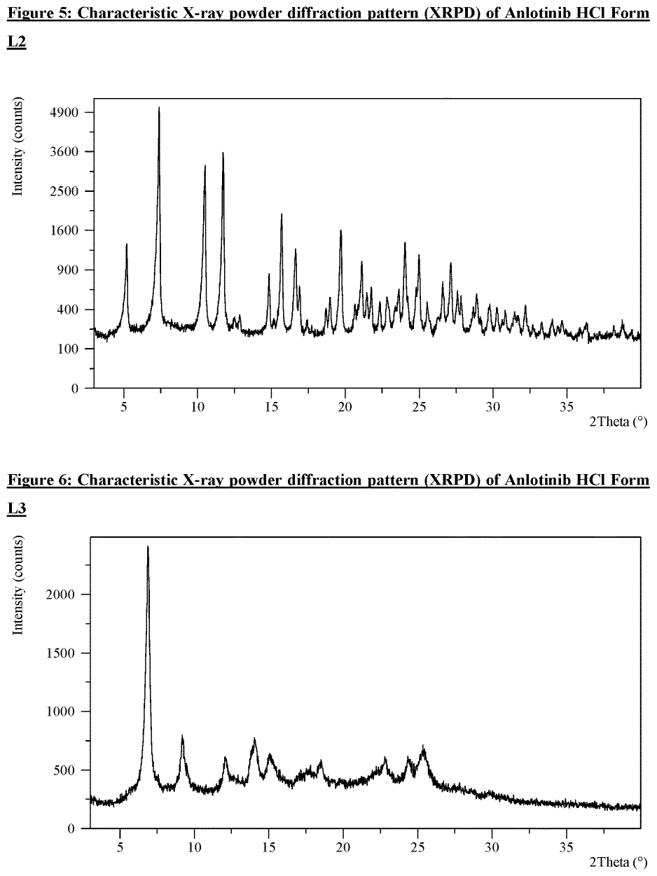

The present disclosure further includes a crystalline polymorph of Anlotinib HCl, designated Form L1. The crystalline Form L1 of Anlotinib HCl may be characterized by data selected from one or more of the following: an X-ray powder diffraction pattern substantially as depicted in FIG. 4; an X-ray powder diffraction pattern having peaks at 9.2, 11.6 and 12.8 degrees 2-theta±0.2 degrees 2-theta; and combinations of these data.

Crystalline Form L1 of Anlotinib HCl may be further characterized by an X-ray powder diffraction pattern having peaks at 9.2, 11.6 and 12.8 degrees 2-theta±0.2 degrees 2-theta, and also having any one, two, three, four or five additional peaks selected from 8.1, 9.8, 14.4, 18.6, 18.9, 19.5 and 24.5 degrees 2-theta±0.2 degrees 2-theta.

In one embodiment of the present disclosure, crystalline Form L1 of Anlotinib HCl is isolated.

Crystalline Form L1 of Anlotinib HCl may be characterized by each of the above characteristics alone/or by all possible combinations, e.g., an XRPD pattern having peaks at 9.2, 11.6 and 12.8 degrees 2-theta±0.2 degrees 2-theta; an XRPD pattern as depicted in FIG. 4, and combinations thereof.

The present disclosure further includes a crystalline polymorph of Anlotinib HCl, designated Form L2. The crystalline Form L2 of Anlotinib HCl may be characterized by data selected from one or more of the following: an X-ray powder diffraction pattern substantially as depicted in FIG. 5; an X-ray powder diffraction pattern having peaks at 7.4, 10.5, 11.7, 15.7 and 19.7 degrees 2-theta±0.2 degrees 2-theta; and combinations of these data.

Crystalline Form L2 of Anlotinib HCl may be further characterized by an X-ray powder diffraction pattern having peaks at 7.4, 10.5, 11.7, 15.7 and 19.7 degrees 2-theta±0.2 degrees 2-theta, and also having any one, two, three, four or five additional peaks selected from 5.2, 14.8, 16.7, 21.1 and 24.0 degrees 2-theta±0.2 degrees 2-theta.

In one embodiment of the present disclosure, crystalline Form L2 of Anlotinib HCl is isolated.

Crystalline Form L2 of Anlotinib HCl may be characterized by each of the above characteristics alone/or by all possible combinations, e.g., an XRPD pattern having peaks at 7.4, 10.5, 11.7, 15.7 and 19.7 degrees 2-theta±0.2 degrees 2-theta; an XRPD pattern as depicted in FIG. 5, and combinations thereof.

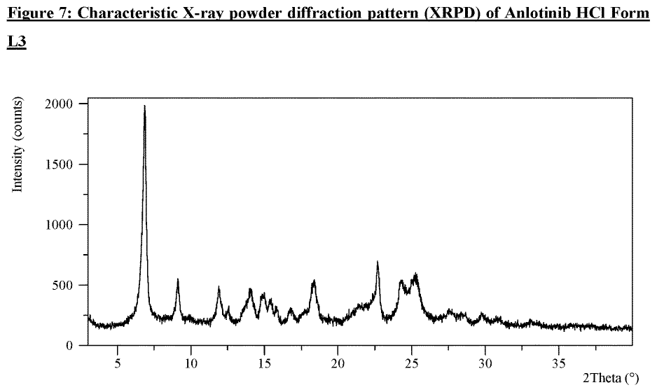

The present disclosure further includes a crystalline polymorph of Anlotinib HCl, designated Form L3. The crystalline Form L3 of Anlotinib HCl may be characterized by data selected from one or more of the following: an X-ray powder diffraction pattern substantially as depicted in FIG. 6; an X-ray powder diffraction pattern having peaks at 6.9, 9.2 and 12.1 degrees 2-theta±0.2 degrees 2-theta; and combinations of these data.

Crystalline Form L3 of Anlotinib HCl may be further characterized by an X-ray powder diffraction pattern having peaks at 6.9, 9.2 and 12.1 degrees 2-theta±0.2 degrees 2-theta, and also having any one, or two additional peaks selected from 14.0 and 22.8 degrees 2-theta±0.2 degrees 2-theta.

In one embodiment of the present disclosure, crystalline Form L3 of Anlotinib HCl is isolated.

In one embodiment of the present disclosure, crystalline Form L3 of Anlotinib HCl is di-HCl salt.

Crystalline Form L3 of Anlotinib HCl may be characterized by each of the above characteristics alone/or by all possible combinations, e.g., an XRPD pattern having peaks at 6.9, 9.2 and 12.1 degrees 2-theta±0.2 degrees 2-theta; an XRPD pattern as depicted in FIG. 6, and combinations thereof.

The present disclosure also encompasses the use of crystalline polymorphs of Anlotinib or salt thereof of the present disclosure for the preparation of pharmaceutical compositions of crystalline polymorph Anlotinib or salt thereof and/or crystalline polymorphs thereof.

The present disclosure includes processes for preparing the above mentioned pharmaceutical compositions. The processes includes combining any one or a combination of the crystalline polymorphs of Anlotinib or salt thereof of the present disclosure with at least one pharmaceutically acceptable excipient.

Pharmaceutical combinations or formulations of the present disclosure contain any one or a combination of the solid state forms of Anlotinib or salt thereof of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Anlotinib or salt thereof and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Anlotinib or salt thereof can be administered. Anlotinib or salt thereof may be formulated for administration to a mammal, in embodiments to a human, by injection. Anlotinib or salt thereof can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Anlotinib or salt thereof and the pharmaceutical compositions and/or formulations of Anlotinib or salt thereof of the present disclosure can be used as medicaments, in embodiments in the treatment of cancer.

The present disclosure also provides methods of treating cancer.

by administering a therapeutically effective amount of any one or a combination of the crystalline polymorphs of Anlotinib or salt thereof of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Sample after being powdered in a mortar and pestle is applied directly on a silicon plate holder. The X-ray powder diffraction pattern was measured with Philips X′Pert PRO X-ray powder diffractometer, equipped with Cu irradiation source=1.54184 Å (Ångström), X'Celerator (2.022° 2θ) detector. Scanning parameters: angle range: 3-40 deg., step size 0.0167, time per step 37 s, continuous scan. Peak positions were determined without using silicon powder as an internal standard.

### EXAMPLES

### Preparation of Starting Materials

Anlotinib or a salt thereof can be prepared according to methods known from the literature, for example as disclosed in U.S. Pat. No. 8,148,532.

### Example 1: Preparation of Anlotinib Form B2

Anlotinib dihydrochloride (10 grams) was dissolved in water (220 mL) at room temperature. Sample was filtrated off over black ribbon filter paper to obtain a clear solution. Sodium hydroxide water solution (1M) was added dropwise until pH=10. The obtained suspension was left to stir overnight. The suspension was filtrated off over blue ribbon filter paper under vacuum. The obtained white powder was dried at 70° C. for 4 hours and then analyzed by XRPD. Anlotinib Form B2 was obtained.

### Example 2: Preparation of Anlotinib Form B4

Anlotinib Form B2 (200 mg) was dissolved in ethyl acetate (19 mL) at 75° C. Heating was turned off and the solution was left to cool to room temperature. Then it was cooled in an ice bath to −6° C. and crystallization started. The suspension was left to stir overnight in cold laboratory (5-10° C.) and then filtrated off over black ribbon filter paper under vacuum. The obtained solid was analyzed by XRPD. Anlotinib Form B4 was obtained.

### Example 3: Preparation of Anlotinib Form B5

Anlotinib Form B2 (500 mg) was suspended in acetone (5 mL) at room temperature. The suspension was left to stir overnight and then filtrated over blue ribbon filter paper under vacuum. The obtained solid was analyzed by XRPD. Anlotinib Form B5 was obtained.

### Example 4: Preparation of Anlotinib HCl Form L1

Anlotinib Form B2 (1 gram) was suspended in 1,4-dioxane (20 mL) at room temperature. 4 mL of hydrogen chloride methanol solution (1.25 M solution, 2.1 eq.) was added dropwise. The obtained suspension was left to stir overnight. The suspension was filtrated off over blue ribbon filter paper under vacuum. The obtained white powder was dried at 40° C. for 2 hours, under vacuum and then for another 2 days at room temperature under vacuum. The obtained powder was analyzed by XRPD. Anlotinib hydrochloride Form L1 was obtained.

### Example 5: Preparation of Anlotinib HCl Form L2

Anlotinib base (600 mg) was suspended in t-butanol/water 100:1 (10.1 mL) at room temperature. 0.5 mL of hydrogen chloride methanol solution (1.25 M solution, 2.7 eq.) was added dropwise. An orange solution was obtained. Crystallization started after a few minutes. The obtained suspension was left to stir for 1 hour and then it was filtrated off over blue ribbon filter paper under vacuum. The obtained powder was analyzed by XRPD. Anlotinib hydrochloride Form L2 was obtained.

### Example 6: Preparation of Anlotinib HCl Form L3

Anlotinib Form B2 (1 gram) was suspended in acetone (20 mL) at room temperature. 1.64 mL of hydrogen chloride 1-butanol solution (3 M solution, 2.1 eq.) was added dropwise. The obtained suspension was left to stir overnight. The suspension was filtrated off over blue ribbon filter paper under vacuum. The obtained white powder was dried at 40° C. for 2 hours under vacuum, then 2 days at room temperature under vacuum and additional 2 hours at 100° C. under vacuum. The obtained powder was analyzed by XRPD. Anlotinib hydrochloride Form L3 was obtained.

### Example 7: Preparation of Anlotinib HCl Form L3

Anlotinib Form B2 (3 grams) was suspended in acetone (60 mL) at room temperature. 9.84 mL of hydrogen chloride 1-butanol solution (3 M solution, 4.2 eq.) was added dropwise. Obtained suspension was left to stir overnight. Suspension was filtrated off over blue ribbon filter paper under vacuum. Obtained white powder was dried at 40° C. for 2 hours under vacuum. Obtained powder was analyzed by XRPD (FIG. 7). Anlotinib dihydrochloride Form L3 was obtained.

1-10. (canceled)

11. A crystalline form of Anlotinib dihydrochloride designated as Form L3, which is characterized by data selected from at least one of:
(i) an X-ray powder diffraction (XRPD) pattern having peaks at 6.9, 9.2 and 12.1 degrees 2-theta±0.2 degrees 2-theta; or
(ii) an XRPD pattern substantially as depicted in FIG. 6.

12. The crystalline form of Anlotinib dihydrochloride according to claim 11, which is characterized by an XRPD pattern having peaks at 6.9, 9.2 and 12.1 degrees 2-theta±0.2 degrees 2-theta, and also having one or two additional peaks selected from 14.0 and 22.8 degrees 2-theta±0.2 degrees 2-theta.

13. A pharmaceutical composition comprising the crystalline form according to claim 11.

14. A pharmaceutical formulation comprising the crystalline form according to claim 11 and at least one pharmaceutically acceptable excipient.

15. A process for preparing a pharmaceutical formulation comprising combining the crystalline form according to claim 11 with at least one pharmaceutically acceptable excipient.
